(19) United States
(12) Patent Application Publication
(10) Pub. No.: US 2004/0033946 A1
(43) Pub. Date: Feb. 19, 2004

(54) Cell damage inhibitor

(76) Inventors: Futoshi Shibasaki, Tokyo (JP); Hiroyuki Uchino, Tokyo (JP); Motohiro Hino, Osaka (JP); Hirohisa Nakada, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/399,044

(22) PCT Filed: Oct. 18, 2001
(86) PCT No.: PCT/JP01/09168

(30) Foreign Application Priority Data
Oct. 19, 2000 (JP) 2000319197

(51) Int. Cl. A61K 38/00

(52) U.S. Cl. 514/12

(57) ABSTRACT

A composition for inhibiting cell damage comprising FR901459 Substance as following formula (I) or its salt in admixture with pharmaceutically acceptable carriers or excipients.

### TECHNICAL FIELD

This invention relates to a pharmaceutical composition for inhibiting cell damage. More particularly, the invention relates to FR901459 Substance as a cell damage inhibitor.

### BACKGROUND ART

In the induction of apoptosis in cells, a dissipation in the membrane potential across the inner membrane of mitochondria is observed in advance of cell death in many instances regardless of the types of cells and/or triggers for induction if cell death. Thus, the derangement of calcium homeostasis and energy metabolism due to death stimuli triggers the opening of the inner membrane channels which are known as permeability transition pores, which, in turn, causes a depression in said potential as well as swelling of mitochondria, thus positively inducing various reactions leading to cell death. Therefore, any drug that suppresses mitochondrial permeability transition is expected to be of use as a therapeutic drug for arresting cell death in tissues in various disease states. It has already been reported that cyclosporin A inhibits the calcium-induced depression in the transmembrane potential and swelling of the mitochondria in vitro, and shows a good neuronal death inhibitory action in ischemic brain models (e.g., WO 96/22104).

### DISCLOSURE OF INVENTION

However, when cyclosporin A, which is an immunosuppressant, is administered for the therapy of cell damage, its immunosuppressive action produces undesirable side effects. Therefore, there has been a standing need for a drug having low risks for immunosuppression and other side effects, and favorable antineuropathic or neuroprotective activity.

The inventors of the present invention made it clear for the first time that FR901459 Substance (JP Kokai H5-271267) inhibits the permeability transition of mitochondria more potently than cyclosporin A, and FR901459 Substance is not only less immunosuppressive than cyclosporin A but the toxic effects of its oral administration in rats are less intense than it is the case with cyclosporin A. Therefore, the activity of FR901459 Substance as elucidated in the present invention is useful for providing a drug showing a higher therapeutic efficacy with a reduced risk for side effects, compared with cyclosporin A, in a number of cytotoxic diseases such as cerebral ischemia, encephalopathy, myocardial infarction and liver diseases.

FR901459 Substance can be produced by fermentation of the strain belonging to fungus Stachybotrys chartaum No.19392. This strain has been deposited with the Patent and Bio-Resource Center (Central 6, 1-1, Higashi 1-chome, Tsukuba-shi, IBARAKI 305-5466 JAPAN) as FERMBP-3364 (deposit date: Apr. 16, 1991). FR901459 Substance can be represented by the following formula (I) differing from that of cyclosporin A.

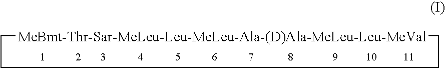

Its chemical formula is C₆₂H₁₁₁N₁₁O₁₃ and its molecular weight is 1,217.

FR901459 Substance can be produced in accordance with the method described in JP Kokai H5-271267 referred to above.

Cell damage is manifested in a number of modes (for example, injury to the cellular tissue, death of the cellular tissue, encephalopathy, diseases arising from generalized or local destruction of the brain, inaction and death) occurring due to a variety of causative factors (for example, ischemia, hypoxemia, cerebrovascular accident, metabolic factor, toxic factor, trauma, surgical factor, compression, hemorrhage, pyrogenic factor, chemical factor, irradiation, vasospasm, neurodegenerative disease, neurodegenerative process, infection, epilepsy, and various causes secondary to such factors). Cell damage entails many sequelae.

In this invention, “inhibition of cell damage” is defined as “an effect leading to suppression or remission of celldamage” and means a protective, resuscitating or regenerative effect on the cellular tissue sustaining cell damage.

“Cell damage inhibitor” is defined, for purposes of this invention, as “a therapeutic or prophylactic drug or a pharmaceutical composition comprising it” to be given in an effective dose to inhibit or relieve cell damage.

This invention discloses the use of FR901459 Substance or a salt thereof in the treatment of the following cell damage-inducing conditions, circumstances or diseases or for providing a therapeutic or prophylactic drug to be used for therapeutic and cytoprotective purposes; for example, FR901459 Substance or its salt can be used for the production of therapeutic or prophylactic drugs to be used therapeutically or cytoprotectively in wounds (bites, closed brain injury, increased intracranial masses and intracranial hypertension, surgical wound), physiological abnormalities (in electrolytes, glucose, vitamins, metabolism, homeostasis, etc.), poisoning (metabolic poisons, toxins, neurotoxins), exposure to radiation (acute and delayed effects), vasospasms, etc., for the treatment of various diseases secondary to, or delayed manifestations of, any of the above conditions, e.g. diseases accompanied by neuropathy of specific systems such as those related to vision, audition, vestibular function, olfaction, etc.; diseases of the brain inclusive of the brain stem and spinal cell tissues or the peripheral nervous system and certain specific diseases (myelitis, myelopathy), etc.; neurodegenerative diseases (Alzheimer 's disease, Parkinson's disease, ALS, Huntington's disease, etc.); infections (herpes virus infection, AIDS associated with cellular sequelae, AIDS myelopathy, etc.; senescence; ischemic neuropathies associated with cerebral thrombosis, cerebral embolism or cerebral hemorrhage; respiratory systemic hypoxia (hypoxic brain in anesthesia; anemia; functional insufficiency of erythrocytes and hemoglobins; hypertension; ischemic liver diseases (cirrhosis etc.); type B or C hepatitis; disturbance of renal blood flow; neuropathies associated with epilepsy or convulsions; and myocardial hypertrophy; or as a liver regeneration promoter; a tissue protectant for the protection of the liver transplant or the prevention of tissue diseases accompanied by cell death; an additive for the preservation of organ grafts; a trichogenic agent; an inhibitor of neurotransmitters; a memory modulating agent; and so forth.

Furthermore, FR901459 Substance or its salt can be administered for the purpose of securing a protective effect on cellular tissues and cell functions before, during, or after occurrence of cell damage.

In this invention, a cell damage inhibitor comprising FR901459 Substance or its salt as an active ingredient can be administered in various solid, semisolid or liquid pharmaceutical preparations formulated with an organic or inorganic carrier or excipient so as to be suited for administration by various routes, e.g. oral; sublingual; buccal; nasal; inhalation; parenteral (intradermal, intraorgan, subcutaneous, intradermal, intramuscular, intraarticular, central venous, hepatic venous, peripheral venous, lymph, cardiac, arterial, selective or highly selective cerebroarterial, or brain parenchymal, or retrograde perfusion into cerebral ventricle from cerebral venous system through a catether); administration into brain or spinal tissue; exposure either direct or under pressure through or onto any cerebrospinal fluid cavity; subarachnoidal, cisternal, subdural or extradural cavity infusion through cisternal paracentesis or lumber puncture; intraocular or periocular instillation inclusive of periocular injection; instillation into the bulb of eye, eyeball structure, or eyeball layer; instillation into the ear inclusive of auditory tube, mastoid air cells, external and internal auditory canals, tympanic membrane, middle ear, internal ear inclusive of cochlear spiral canal ganglion, labyrinth, etc.; or administration into intestinal tract, rectum, vagina, ureter, or urinary bladder. In intrauterine and perinatal indications, too, it can be administered into the mother's blood vessels or organs inclusive of uterus, uterine cervix and vagina, embryo, fetus, neonate, and association tissues and amnion, umbilical cord, umbilical artery and vein, placenta and the like spaces. Although parenteral administration is preferred, the route should be varied according to the patient's condition.

FR901459 Substance or its salt can be administered alone as a therapeutic drug but it is also a good practice to use it as part of a formulation. The “cell damage inhibitor” of this invention can be used in the form of a solid, semisolid or liquid pharmaceutical preparation containing it in combination with at least one or several suitable organic or inorganic carriers or excipients or in admixture with other pharmacologically active substances. For example, the active ingredient can be mixed with a nontoxic carrier in routine use in the pharmaceutical field and provided as granules, tablets, pellets, troches, capsules, suppositories, creams, ointments, aerosols, inhalant powders, liquid dosage forms such as injectable solutions, emulsions or suspensions; preparations for oral intake; eye-drops; and other dosage forms suitable for administration. Where necessary, formulating additives such as stabilizers, thickeners, wetting agents, hardeners, coloring agents, etc.; flavors and buffers; and other routine additives can be incorporated in the above preparation.

In the “cell damage inhibitor” of the invention is formulated a sufficient amount of FR901459 Substance or its salt to insure the expected cell damage inhibitory effect according to the course or status of illness.

The therapeutically effective dose of FR901459 Substance or its salt varies with the patient's age and condition and depends also on dosage form, mode of administration, stage of illness and administration interval but the therapeutic drug is usually formulated in a proportion of 0.1 through 90% based on the total weight of the composition. For the inhibition of cell damage, 0.0001 through 50 mg/day per kg bodyweight, preferably 0.001 through 25 mg, can be administered parenterally or 0.001 through 100 mg/day per kg body weight, preferably 0.01 through 60 mg, can be administered enterally. However, there are cases in which the upper limit mentioned above must be exceeded to achieve the expected therapeutic benefit.

Suitable salts of FR901459 Substance are pharmaceutically acceptable, ordinary nontoxic salts. Thus, for example, there can be mentioned salts with bases and acid addition salts, for example salts with inorganic bases (e.g. alkali metal salts such as sodium salt, potassium salt, etc.; alkaline earth metal salts such as calcium salt, magnesium salt, etc.; ammonium salts), salts with organic bases (e.g. organic amine salts such as triethylamine salt, diisopropylethylamine salt, pyridine salt, picoline salt, ethanolamine salt, triethanolamine salt, dicyclohexylamine salt, N,N′-dibenzylethylenediamine salt, etc.), inorganic acid addition salts (e.g. hydrochloride, hydrobromide, sulfate, phosphate, etc.), organic carboxylic or sulfonic acid addition salts (e.g. formate, acetate, trifluoroacetate, maleate, tartrate, gluconate, fumarate, methanesulfonate, benzenesulfonate, toluenesulfonate, etc.), and salts with basic or acidic amino acids (e.g. arginine, aspartic acid, glutamic acid, etc.).

FR901459 Substance or its salt includes solvated compounds (e.g. hydrate, ethanolate, etc.).

FR901459 Substance or its salt includes crystalline and non-crystalline forms.

This invention relates to the use of the therapeutic drug of the invention under the conditions set forth throughout this specification. This invention, therefore, encompasses all relevant advertisements, labels, packages, data sheets, advertising inserts, product specifications, advertising materials, characters, pamphlets, magazines, books; conversations and communications using various media such as facsimile, telephone, photograph, radio, video, television, film, internet, e-mail, etc.; computer-aided presentation of information, proposals concerning clinical trials, and protocols for clinical studies using the therapeutic drug of the invention with regard to the inhibition of cell damage, among others.

The patent specifications and publications mentioned herein are incorporated by reference in this specification.

The following Examples are intended to illustrate this invention in further detail and should by no means be construed as defining the scope of the invention.

### EXAMPLE 1

Effects of FR901459 Substance and Cyclosporin A on the Calcium-induced Swelling of Mitochondria Isolated from the Brain

When the cells constituting a living tissue are subjected to a cytotoxic stress (oxygen deficiency, nutritional factor deficiency, etc.), the cytosolic calcium is increased to induce opening of the permeability transition pores of mitochondria, whereupon the inorganic ions, water and biological molecules around the mitochondria find their way into the mitochondria to cause a membrane potential depression and swelling of the mitochondria, with cell death ensuing. This reaction can be reproduced by isolating mitochondria from a living tissue and elevating the calcium concentration in a suspension of the mitochondria. The swelling of mitochondria was monitored by measuring the intensity of scattered light (540 nm) (light at 540 nm is scattered through an angle of 90° C.) (Perkin-Elmer LS-50B fluorescence spectrometer). Based on the condition prior to addition of CaCl₂ (intensity of scattered light: ca 1.3) and the condition after forced swelling due to addition of alamethicin (40 μg/mg mitochondrial protein) (intensity of scattered light: ca 0.4), the rates of inhibition of mitochondrial swelling at various concentrations of cyclosporin A and FR901459 Substance were determined.

As a result, whereas cyclosporin A caused an approximately 50% inhibition at about 250 nM, FR901459 Substance caused a 50% inhibition at 25 nM, indicating that FR901459 Substance is about 10 times as active as cyclosporin A.

When cyclosporin A or FR901459 Substance is administered for the therapy of cell damage, the immunosuppressive action of each drug may produce undesirable side effects. That FR901459 Substance is less immunosuppressive and, as a therapeutic drug, has more favorable properties than cyclosporin A is demonstrated in Example 2.

### EXAMPLE 2

Effects of FR901459 Substance and Cyclosporin A on the MLR Reaction in Mice

In accordance with the method reported by Izumi et al. in Cancer Res., Vol. 46 (1960-1965) (1986), spleen cells were harvested from female Balb/c (H-2^{d}) mice and female C57BL/6 (H-2^{b}) mice, respectively, to prepare a cell suspension. A flat-bottomed microtiter plate was seeded with the suspension of 5×10⁵ responder cells derived from Balb/c and 2.5×10⁵ stimulator cells harvested from C57BL/6 and treated with X-rays in RPMI medium (10% fetal calf serum, 50 μM 2-mercaptoethanol, 100U/ml penicillin, 100 μg/ml streptomycin added), 100 μl/well. The cells were grown at 37° C. in an humidified (water vapor-saturated) atmosphere of 5% CO₂, 95% air for 72 hours. During the last 4 hours, 18.5 kBq of ³H-labeled thymidine (New England Nuclear, Boston, Mass.) was added to the medium. Then, the cells were recovered on the glass fiber strip of a microharvestor and the degree of cell growth was estimated from the radioactivity. The results are shown in Table 1.

TABLE 1
Inhibitory effects of FR901459 Substance and cyclosporin A on
MLR in rats

| | Mean ± SE | Inhibition rate |
|---|---|---|
| Drug | (cpm) | (%) |
| Control | 7174 ± 166 | |
| Responder alone | 1390 ± 61** | |
| Stimulator alone | 44 ± 8** | |
| FR901459 Substance (0.3 ng/ml) | 7886 ± 318 | −12.4 |
| FR901459 Substance (1.0 ng/ml) | 7762 ± 323 | −10.2 |
| FR901459 Substance (3.2 ng/ml) | 7789 ± 529 | −10.7 |
| FR901459 Substance (10 ng/ml) | 6201 ± 252* | 17.0 |
| FR901459 Substance (32 ng/ml) | 4089 ± 212** | 53.7 |
| FR901459 Substance (100 ng/ml) | 1514 ± 160** | 98.6 |
| FR901459 Substance (320 ng/ml) | 527 ± 29** | 115.8 |
| Cyclosporin A (0.3 ng/ml) | 7041 ± 381 | 2.3 |
| Cyclosporin A (1.0 ng/ml) | 6736 ± 238 | 7.6 |
| Cyclosporin A (3.2 ng/ml) | 6129 ± 353* | 18.2 |
| Cyclosporin A (10 ng/ml) | 4906 ± 417** | 39.5 |
| Cyclosporin A (32 ng/ml) | 1970 ± 163** | 90.7 |
| Cyclosporin A (100 ng/ml) | 264 ± 20** | 120.4 |
| Cyclosporin A (320 ng/ml) | 91 ± 9** | 123.4 |

*p < 0.05, compared with control (Dunnett's multi-comparison)
**p < 0.01, compared with control (Dunnett's multi-comparison)

As shown in Table 1, whereas FR901459 Substance shows statistically significant MLR-inhibitory activity at 10 ng/ml and higher concentrations, cyclosporin A showed significant inhibitory activity at 3.2 ng/ml and higher concentrations.

It is apparent from Experimental Examples 1 and 2 that while FR901459 Substance is about 10 times as potent as cyclosporin A in the effect of suppressing mitochondrial damage (permeability transition) occuring in the induction of apoptosis, its immunosuppressive action is about 3-fold as weak as

cyclosporin A.

Therefore, FR901459 Substance is useful as a favorable cell damage inhibitor, exhibiting inhibitory effects on cell damage, particularly neuronal damage. Furthermore, FR901459 Substance is considered to find application as an effective tissue protectant in all tissue diseases accompanied by cell death, such as postischemic reperfusion disorders of the liver and heart, hemodynamic distrubance of the kidney, and so forth.

The patents, patent applications and publications cited herein are incorporated by reference.

1. A pharmaceutical composition for inhibiting cell damage comprising FR901459 Substance or its salt in admixture with pharmaceutically acceptable carriers or excipients.

2. Use of FR901459 Substance or its salt for the manufacture of a cell damage inhibitor.

3. A method of inhibiting cell damage, which comprises administering FR901459 Substance or its salt to a human being or an animal.

4. A composition of claim 1, in which the cell damage is neuronal damage.

5. A composition of claim 1, in which the cell damage is neurodegerative diseases.

6. A composition of claim 1, in which the cell damage is ischemic neuropathies.

7. A commercial package comprising the pharmaceutical composition of FR901459 Substance and a written matter associated therewith, wherein the written matter states that the pharmaceutical composition can or should be used for inhibiting cell damage.

8. An article of manufacture, comprising packaging material and the FR901459 Substance contained within said packaging material, wherein FR901459 Substance is effective for inhibiting cell damage, and wherein said packaging material comprises a label or a written material which indicates that FR901459 can or should be used for inhibiting cell damage.
